(19) United States
(12) Patent Application Publication
(10) Pub. No.: US 2009/0123543 A1
(43) Pub. Date: May 14, 2009

(54) PHARMACEUTICAL COMPOSITIONS

(75) Inventors: Pratibha S. Pilgaonkar, Mumbai (IN); Maharukh T. Rustomjee, Mumbai (IN); Anilkumar S. Gandhi, Mumbai (IN); Paras R. Jain, Mumbai (IN)

Correspondence Address: MERCHANT & GOULD PC, P.O. BOX 2903, MINNEAPOLIS, MN, 55402-0903, US

(73) Assignee: Rubicon Research Private Limited, Mumbai (IN)

(21) Appl. No.: 12/159,506

(22) PCT Filed: Jan. 2, 2007
(86) PCT No.: PCT/IN2007/000004

§ 371 Date: Oct. 2, 2008

(30) Foreign Application Priority Data
Jan. 2, 2006 (IN) 3/MUM/2006

(51) Int. Cl. A61K 9/36; A61K 31/41; A61K 31/495; A61P 9/12; A61K 9/32; A61K 31/40

(52) U.S. Cl. 424/480; 514/381; 514/249; 514/423; 424/482

(57) ABSTRACT

A novel solid oral dosage form comprising a therapeutically effective amount of hydrophobic pharmacological active ingredient and at least one particle separating agent preferably selected from a class of wetting agents, prepared without or with minimum amount of a disintegrating agent. The hydrophobic pharmacological active ingredient active ingredient belongs to the class of angiotensin receptor blocking agents preferably is valsartan optionally in combination with hydrochlorothiazide. The active ingredient may also be a class of 3-hydroxy-3-methylglutaryl coenzyme A (HMG CoA) reductase inhibitors preferably atorvastatin. The ratio of hydrophobic active ingredient to particle separating agent is about 20:1 to about 1:20. The process for the preparation of the novel solid oral dosage form comprises treating a hydrophobic active ingredient with at least one particle separating agent, and incorporating the treated hydrophobic active ingredient into a solid dosage form.

### FIELD OF INVENTION

The present invention relates to solid oral dosage forms of hydrophobic active ingredients. The invention particularly relates to solid oral dosage forms of hydrophobic active ingredients prepared by treating pharmaceutically effective amounts of the active ingredient with at least one “particle separating agent” and then incorporating the said treated active ingredient into a solid dosage form.

The present invention more particularly relates to selective use of the particle separating agent in the solid oral dosage forms of hydrophobic active ingredients thereby giving desired disintegration time and dissolution profile without or with a minimum amount of traditional disintegrants.

The present invention further particularly relates to selective use of the particle separating agent in the solid oral dosage forms of hydrophobic active ingredients belonging to the class of angiotensin receptor blockers (ARBs) and 3-hydroxy-3-methylglutaryl coenzyme A (HMG CoA) reductase inhibitors

### BACKGROUND OF INVENTION

Hydrophobic active ingredients pose considerable challenges to the formulation scientist. Apart from poor aqueous solubility, they usually have tendency to aggregate, poor flow and static charges. Their low aqueous solubility results in slow disintegration or dissolution leading to reduced absorption of the drug and ultimately may lead to poor bioavailability. Moreover, when such poorly soluble drugs are formed into tablets, the processes employed for preparation of tablets may further reduce the disintegration or dissolution.

A frequently used method to overcome such problems is to finely grind or micronize drug substance so as to reduce their particle size to a range of about 100 microns or less. A major disadvantage of such grinding methods is the resulting tendency of the milled particles to agglomerate and the formation of an electrostatic charge on their surface, which leads to poor flow and wetting properties.

One of the commonly employed methods to resolve this agglomeration is to use disintegrants, which helps in breaking up of larger particles/granules into finer particles. PCT applications WO 8705804 and WO 0015196 describes use of more than 10% of disintegrants for hydrophobic drugs in order to improve disintegration and in turn the dissolution. A combination of disintegrants are employed in U.S. patent application 2004/0028741 and WO 05089710 in order to achieve a desired dissolution profile for hydrophobic active ingredient.

In case of another hydrophobic drug belonging to class of ARB, valsartan, similarly higher concentrations of disintegrants are employed. PCT application WO 9749394 claim a solid oral dosage form wherein valsartan is present in an amount more than 35% by weight based on total weight of the solid dosage form. The formulation prepared by roll compaction process, contains a superdisintegrant, particularly crosslinked polyvinylpyrrolidone (crospovidone), in an amount from 10 to 30% by weight of formulation. Another U.S. patent application 20020132839 claims use of 20-80% disintegrant for valsartan.

However use of higher amounts of disintegrant in a dosage form is not desirable for a number of reasons. Disintegrants upon exposure to fluid environment, have a tendency to absorb large amount of moisture, a property that is desired for its functionality. Absorption of moisture on the other hand during processing such as wet granulation and aqueous film coating would result in a negative effect of its performance. Moreover the coated tablets would have undesirable surface defects such as orange peel effect due to the excessive uptake of water by the disintegrant.

Moisture pick-up during shelf life by a dosage form containing high amounts of disintegrants can result in altered disintegration time leading to compromise in in-vitro performance of the dosage form which may further result in suboptimal in-vivo behavior. Therefore a costlier pack such as Alu-alu blister may be required

The increase in disintegrant concentration also means increased tablet weight, which is usually not desired as larger tablets are usually difficult to swallow. Further use of large amount of superdisintegrant may adversely affect the compressibility of the dosage form and may cause surface to appear rough.

Another approach to avoid the problems associated with micronized hydrophobic actives is to employ large amounts of diluents. Diluents as the name suggest would dilute the hydrophobic active ingredient and help in their processing. U.S. Pat. No. 6,126,971 describes immediate release formulations of atorvastatin a HMG CoA reductaste inhibitor, which also is a hydrophobic active, stabilized by alkaline earth metal salts. Product based on this patent i.e. lipitor contains 80 mg of atorvastatin in 1200 mg tablet, indicating use of large amount of diluents. Another PCT application WO 04110431 indicates that atorvastatin is not very stable in the presence of the disintegrant croscarmellose sodium. Despite this instability croscarmellose is employed at almost 1:1 ratio (Drug:Disintegrant) in the marketed formulation to achieve the desired disintegration and dissolution. The limitation of this formulation therefore is larger tablet weight and need of stabilizers and other approaches to stabilize the active ingredient.

Thus the above approaches employed for formulation of hydrophobic active ingredients not only increase the tablet weight and the cost of the formulation but also makes the manufacturing process labor intensive and time consuming. Moreover in several instances, it leads to a formulation which fails in the specifications during accelerated stability studies unless packed in expensive packs. Still no attempts have been made in the prior art to improve the processability of these hydrophobic actives and to solve the problem of need of a large amount of a disintegrating agent. Thus there is a long felt need in the prior art to develop formulations of hydrophobic active ingredient containing minimum amount of disintegrants. The inventors of the present invention have addressed the problem by using agents, hereafter described as particle separating agents.

The inventors have surprisingly found that particle separating agents help to achieve the desired disintegration time of the solid dosage formulations of hydrophobic active ingredients without or with a minimum amount of disintegrating agent. The treatment of hydrophobic active ingredient with a particle separating agent dramatically improves their surface properties such as wettability, reduction in static charge, flow etc. Due to this improved surface properties particles have tendency to rapidly separate from each other thus improving their processability and resulting in reduced disintegration time and improved dissolution profile.

### OBJECT OF INVENTION

It is an object of the present invention to improve the surface properties of hydrophobic active ingredients by treating them with at least one particle separating agent.

It is a further object of the present invention to provide hydrophobic active ingredient treated with a particle separating agent as an solid oral formulation without a traditional disintegrant.

It is another object of the present invention to provide hydrophobic active ingredient treated with a particle separating agent as an solid oral formulation with a minimum amount of a traditional disintegrant.

It is yet another object of the present invention to provide hydrophobic active ingredient treated with a particle separating agent as an solid oral formulation which gives the desired processability, disintegrating time and dissolution profile.

It is further object of the present invention to provide a process for the preparation of a solid oral formulation of a hydrophobic active ingredient in which the hydrophobic active ingredient is treated with at least one particle separating agent.

It is a further object of the present invention to provide a hydrophobic active ingredient selected from a class of ARB, treated with a particle separating agent as an oral solid formulation without or with a minimum amount of a traditional disintegrant, but which gives the desired processability, disintegrating time and dissolution profile.

It is yet another further object of the present invention to provide a simple process for preparing oral solid tablet dosage forms comprising pharmaceutically effective amounts of an ARB or pharmaceutically acceptable salt thereof in combination with pharmaceutically effective amounts of hydrochlorthiazide that gives desired disintegration time and dissolution profile whilst including no or a minimum amount of a traditional disintegrant.

It is a further object of the present invention to provide a hydrophobic active ingredient selected from a class of HMG CoA reductase inhibitor, treated with a particle separating agent as an oral solid formulation without or with a minimum amount of a traditional disintegrant, but which gives the desired processability, disintegrating time and dissolution profile.

It is a further object of the present invention to provide valsartan treated with a particle separating agent as an oral solid formulation without or with a minimum amount of a traditional disintegrant, but which gives the desired processability, disintegrating time and dissolution profile.

It is a further object of the present invention to provide atorvastatin treated with a particle separating agent as an oral solid formulation without or with a minimum amount of a traditional disintegrant, but which gives the desired processability, disintegrating time and dissolution profile.

### SUMMARY OF INVENTION

According to an aspect of the present invention there is provided an oral solid dosage form comprising at least one particle separating agent in combination with a hydrophobic active ingredient.

According to a further aspect of the present invention there is provided a process for the preparation of an oral solid form comprising the steps of:

(a) treating a hydrophobic active ingredient with at least one particle separating agent, and
(b) incorporating the treated hydrophobic active ingredient into a solid dosage form.

### DETAILED DESCRIPTION OF INVENTION

Hydrophobic actives are characterized by a set of physical properties such as

1. High log P (oil/water partition coefficient) value

2. Low aqueous solubility

3. High dose to solubility ratio

Due to their hydrophobicity these agents do not form hydrogen bond with water resulting in poor wettability of the tablet and delayed disintegration leading to low dissolution. Further, due to poor wettability of drug particles, they remain in the form of larger aggregates rather than unit particles. Therefore, tablet formulations of such actives require large quantity of disintegrating agent in intimate contact with drug particles to promote the disintegration into unit particles. The inventors of the present invention have addressed the problem of the need for large amounts of traditional disintegrating agents required in oral solid formulations of hydrophobic active ingredients.

It has now been surprisingly found that treatment of hydrophobic active ingredient with a particle separating agent dramatically improves their surface properties and the treated particles have tendency to rapidly separate from each other in aqueous media in the absence of or in presence of a minimum amount of a traditional disintegrant. Accordingly, the present inventors have carried out rigorous experiments for the selection of particle separating agents, which achieve the desired disintegration time of these solid dosage formulations of hydrophobic active ingredients.

The oral solid formulation of the present invention comprises at least one particle separating agent in combination with an hydrophobic active ingredients and excipients.

The term ‘hydrophobic active ingredients’ as used herein includes agents that have a lack of affinity for water and precipitate at concentration greater than 10 mg/ml in any of the physiologically relevant media such as 0.1N HCl, pH 4.5 acetate buffer, Ph 6.8 phosphate buffer, simulated gastric fluid in fasted or fed state, simulated small intestinal fluid etc.

The term ‘traditional disintegrating agent’ as used herein includes agents that are included a tablet or capsule blend to aid in the break up of the compacted mass when it is put into a fluid environment. More particularly the term refers to superdisintegrants such as croscarmellose sodium, crospovidon etc which are usually employed at relatively lower concentrations.

The term ‘large amount of traditional disintegrating agent’ as used herein means use of more than 10% of a traditional disintegrating agent (which is more than the conventional usage level) in a formulation

The term ‘minimum amount of traditional disintegrating agent’ as used herein means use of less than 10%, preferably less than 7.5% of a traditional disintegrating agent in a formulation.

The term “particle separating agent” used in the present invention is defined as an agent, which promotes separation of hydrophobic active ingredient's particles from each other (which otherwise tend to form agglomerates), by changing their surface properties.

### Hydrophobic Active Ingredients

Drugs that are poorly soluble in water can have pharmaceutical efficacy in a number of therapeutic and diagnostic imaging areas. Non-limiting classes of compounds that are useful in this invention can be selected include anesthetic agents, ace inhibiting agents, antithrombotic agents, anti-allergic agents, antibacterial agents, anticoagulant agents, anticancer agents, antidiabetic agents, antihypertension agents, antifungal agents, antiinflammatory agents, antimigraine agents, antiparkinson agents, antirheumatic agents, antithrombins, antiviral agents, beta blocking agents, bronchospamolytic agents, calcium antagonists, cardiovascular agents, cephalosporins, contraceptive agents, diuretic agents, fibrinolytic agents, growth hormones, immunosupressants, lipid-lowering agents, neurologic agents, prostacyclins, prostaglandins, psycho-pharmaceutical agents, protease inhibitors, vasodilating agents, vitamins and the like.

Exemplary hydrophobic drugs belong to the class of antihypertensive agents such as angiotensin receptor blockers and lipid lowering agents such as 3-hydroxy-3-methylglutaryl coenzyme A (HMG CoA) reductase inhibitors.

The particular hydrophobic active ingredient belonging to the class of ARBs include valsartan, candesartan, eprosartan, irbesartan, losartan, olmesartan, telmisartan, and pratosartan.

The particular hydrophobic active ingredient belonging to the class of HMG CoA reductase inhibitors include atorvastatin, lovastatin, pravastatin, simvastatin, mevastatin, cerivastatin, fluvastatin and the like.

Most preferred pharmacologically active ingredient among these are valsartan and atorvastatin

The active ingredient of the invention may be present in crystalline or amorphous form or as a solid solution or dispersion form. The crystalline form may have different polymorphs. All different polymorphs, solvates, hydrates, salts are within the purview of this invention.

The pharmacologically active ingredient or ingredients are present in a therapeutically effective amount, which is an amount that produces the desired therapeutic response upon oral administration and can be readily determined by one skilled in the art. In determining such amounts, the particular pharmacologically active ingredient being administered, the bioavailability characteristics of the pharmacologically active ingredient, the dose regime, the age and weight of the patient, and other factors must be considered, as known in the art. However in the formulation the active ingredient may be present in an amount 5-80%, preferably 10-70% and more preferably 15-65% by weight of the composition.

In certain embodiments of the present invention, one or a combination of more than one pharmacologically active ingredients can also be employed. For instance, in a dosage form of the present invention, in addition to an ARB, one or more, for example two, furthermore three, active ingredients, as specified according to the present invention, can be combined. The therapeutic agents, which may be combined with an ARB include, but are not limited to, anti-hypertensive agents particularly hydrochlorothiazide, anti-obesity agents, anti-diabetic agents, beta-blockers, inotropic agents and hypolipidemic agents.

### Particle Separating Agent:

Particle separating agents modify the surface properties of the hydrophobic active ingredient and make them amenable for convention processing and at the same time achieve the desired disintegration and dissolution without or with a minimum amount of a traditional disintegrating agent. The said surface properties may include wettability, flow property, static charge, hydrophobicity and compressibility.

According to present invention the particle separating agent is selected from a group of substances more commonly known as wetting agents

Wetting agent, usually a surface active agent, reduces the surface tension of a liquid and therefore increases its adhesion to a solid surface. Improved wettability is observed as a lower contact angle between the solid and liquid. A wetting agent usually consists of a molecule with a hydrophilic (water attracting) group at one end and a hydrophobic (water repelling, and therefore oil attracting) group at the other.

The wetting agents for the purpose of the present invention may be selected from hydrophilic surfactants or lipophilic surfactants or mixtures thereof. The surfactants may be anionic, nonionic, cationic, and amphiphilic surfactants.

The hydrophilic non-ionic surfactants may be selected from the group comprised of, but not limited to: polyethylene glycol sorbitan fatty acid esters and hydrophilic transesterification products of a polyol with at least one member of the group consisting of triglycerides, vegetable oils, and hydrogenated vegetable oils preferably glycerol, ethylene glycol, polyethylene glycol, sorbitol, propylene glycol, pentaerythritol, or a saccharide, d-α-tocopheryl polyethylene glycol 1000 succinate and d-l-tocopherol and its acid salts such as succinate, adipate, etc.

The ionic surfactants may be selected from the group comprised of, but not limited to: alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

The lipophilic surfactants may be selected from the group consisting of, but not limited to: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; PEG sorbitan fatty acid esters, PEG glycerol fatty acid esters, polyglycerized fatty acid, polyoxyethylene-polyoxypropylene block copolymers, sorbitan fatty acid esters; and mixtures thereof.

Preferably the wetting agent may be selected from PEG-20-glyceryl stearate (Capmul® by Abitec), PEG-40 hydrogenated castor oil (Cremophor RH 40® by BASF), PEG 6 corn oil (Labrafil® by Gattefosse), lauryl macrogol-32 glyceride (Gelucire 44/14® by Gattefosse) stearoyl macrogol glyceride (Gelucire 50/13® by Gattefosse), polyglyceryl-10 mono dioleate (Caprol® PEG 860 by Abitec), Propylene glycol dioctanoate (Captex® by Abitec) Propylene glycol caprylate/caprate (Labrafac® by Gattefosse), Glyceryl monooleate (Peceol® by Gattefosse), Glycerol monolinoleate (Maisine® by Gattefosse), Glycerol monostearate (Capmul® by Abitec), PEG-20 sorbitan monolaurate (Tween 20® by ICI), PEG-4 lauryl ether (Brij 300 by ICI), Sucrose distearate (Sucroester 7® by Gattefosse), Sucrose monopalmitate (Sucroester 15® by Gattefosse), polyoxyethylene-polyoxypropylene block copolymer (Lutrol® series BASF), polyethylene glycol 660 hydroxystearate, (Solutol® by BASF), Sodium lauryl sulphate, Sodium dodecyl sulphate, Dioctyl suphosuccinate, L-hydroxypropyl cellulose, hydroxylethylcellulose, hydroxy propylcellulose, Propylene glycol alginate, sodium taurocholate, sodium glycocholate, sodium deoxycholate, betains, polyethylene glycol (Carbowax® by DOW), d-α-tocopheryl polyethylene glycol 1000 succinate (Vitamin E TPGS® by Eastman) and mixtures thereof.

A more preferred wetting agent may be selected from PEG-40 hydrogenated castor oil (Cremophor RH 40® by BASF-HLB-13), lauryl macrogol-32 glyceride (Gelucire 44/14®) by Gattefosse-HLB-14) stearoyl macrogol glyceride (Gelucire 50/13® by Gattefosse-HLB-13), PEG-20 sorbitan monolaurate (Tween 20® by ICI-HLB-17), PEG-4 lauryl ether (Brij 30® by ICI-HLB-9.7), polyoxyethylene-polyoxypropylene block copolymer (Lutrol® series BASF having different HLB ranging from 15-30), polyethylene glycol 660 hydroxystearate, (Solutol® by BASF), Sodium lauryl sulphate (HLB-40), polyethylene glycol (Carbowax®) by DOW), d-α-tocopheryl polyethylene glycol 1000 succinate. (Vitamin E TPGS® by Eastman-HLB-15) and mixtures thereof.

In the composition the hydrophobic active ingredient and one or more particle separating agent may be employed in different ratios. The selection of ratio depends upon the desired improvement in surface properties and the type of particle separating agent employed. It is contemplated within the scope of the invention that the ratio of hydrophobic active ingredient to particle separating agent can range from about 20:1 to about 1:20. The preferred ratio of the hydrophobic active ingredient to particle separating agent ranges from about 10:1 to about 1:10. The most preferred ratio being about 5:1 to about 1:5.

A combination of particle separating agent may also be included wherein the total amount of particle separating agent employed is maintained in the above-mentioned ratios.

In the composition, the hydrophobic active ingredient may be present in the form of physical blend, granular form, semi solid mixture, solid solution or complex with the particle separating agent.

Different non-limiting processes may be employed for the treatment of a hydrophobic active ingredient with particle separating agent. It is contemplated within the scope of the invention that the processes may include treatment using melt granulation or solvent treatment or physical mixing or complexation method. In case of melt granulation, the particle separating agent (s) is (are) melted and the hydrophobic active ingredient is added and mixed with the molten mass effectively, allowed to solidify and the granules are separated from each other.

In another illustrative embodiment of this system the hydrophobic active ingredients granulated using molten particle separating agent. In some cases the hydrophobic active ingredient and particle separating agent both may be melted together and cooled to room temperature. In some cases the molten mixture of the active ingredient and the particle separating agent can be filled into capsule and solidify inside the capsule.

In using a solvent treatment method, either the particle separating agent or the hydrophobic active ingredient, or both are dissolved in a solvent and the solvent is then evaporated. When particle separating agent is dissolved in a solvent, the solution is employed for treating the hydrophobic active ingredient. The resultant mass is a blend of the hydrophobic active ingredient and particle separating agent, such that the surface properties of the hydrophobic active ingredient are improved. Solvent employed in this system may be aqueous or pharmaceutically acceptable non-aqueous solvents.

In case of physical mixing, the hydrophobic active ingredient and particle separating agent are intimately mixed.

It is contemplated within the scope of the invention that a combination of hot melt process, physical mixing and solvent treatment method can be employed. In this case the hydrophobic active ingredient may be initially granulated with one or more molten particle separating agent which can be further treated with same or different excipients in a solvent or by simple physical mixing or vice versa. It is also contemplated within the scope of the invention that any process known in the art suitable for improving surface properties in general may be employed for the purpose of this invention.

According to this invention, melt granulation and intimate physical mixture are the most preferred methods for treatment of a hydrophobic active ingredient.

### Excipients:

The dosage form according to the invention may include other excipients conventionally known in art such as fillers, binders and lubricants. Fillers such as lactose monohydrate, microcrystalline cellulose, dicalcium phosphate or the like may be used. Binders like polyvinyl pyrrolidone (PVP), copovidone or the like may be used. Lubricants such as Aerosil-200, magnesium stearate, sodium steryl fumarate and hydrogenated vegetable oils and triglycerides of stearic acid, palmitic acid or the like may be utilized. The formulation may also include a basifying agent that raises pH in the immediate environment of the substrate to which it is applied. The said basifying agents belong to class of metal hydroxides, basic salts of mono-, di-, tri-protic acids, or basic amino acids.

Basifying agents for the purpose of this invention may be selected from sodium hydroxide, potassium hydroxide, calcium carbonate, sodium bicarbonate, potassium bicarbonate, sodium carbonate, potassium carbonate, mono, di or tri calcium citrate, glucosamine, arginine, meglumine and the like.

The disintegrating agent may be selected from a group but not limited to the following: starch, sodium starch glycolate, pregelatinised starch, crosslinked poly vinyl pyrrolidone, cross linked carboxy methyl cellulose, ion exchange resin and the like The disintegrant may be present in an amount ranging from about 0% to about 10%, more preferably about 0% to about 7.5% and most preferably from about 0 to about 5% by weight based on the total weight of the composition.

A therapeutically effective amount of a hydrophobic active ingredient, in free or a pharmaceutically acceptable salt form is supplied as a suitable unit dosage form, e.g. a tablet.

The proposed technique of improving wettability and then formulating into a dosage form without using excessive amounts of disintegrating agent can also be applied for other dosage forms for example, powders of given doses packaged in sachets, suspensions, gelatin capsules, soft gelatin capsules, semisolid dosage forms as well as other drug delivery systems.

The dosage form of the present invention is a solid dosage form, preferably a tablet, which may vary in shape including but not limited to oval, triangle, almond, peanut, parallelogram, pentagonal. It is contemplated within the scope of the invention that the dosage form can be encapsulated.

The dosage form of the present invention can exist in various pharmaceutical dosage forms, including in particular: tablets which disintegrate in stomach, tablets which can disintegrate in the mouth, tablets which can disintegrate by effervescence in a liquid (water), tablets which can disintegrate in a liquid (water), coated tablets.

Tablets in accordance with the invention may be manufactured using conventional techniques of common tableting methods known in the art such as direct compression, wet granulation, dry granulation and extrusion/melt granulation. This also includes pellets made using extrusion spheronization process and compressed into tablets.

In one illustrative embodiment according to the invention, the dosage form may be optionally coated. Surface coatings may be employed for aesthetic purposes or for dimensionally stabilizing the compressed dosage form or for enteric release or for controlled release. The surface coating may be any conventional coating which is suitable for enteral use. The coating may be carried out using any conventional technique employing conventional ingredients. A surface coating can for example be obtained using a quick-dissolving film using conventional polymers such as hydroxypropyl methyl cellulose, hydroxypropyl cellulose, carboxymethyl cellulose, polyvinyl alcohol poly methacrylates or the like.

In one illustrative embodiment according to the invention, the hydrophobic active ingredient treated with a particle separating agent may be incorporated in liquid form into a capsule. The hydrophobic active ingredient mixed with a molten excipient is filled into capsules with or without other excipients. The content of the capsule may remain in liquid or semisolid state during shelf life or the liquid filled into capsule may set to form a solid mass inside capsule. Optionally other excipients such as disintegrants, lubricants, diluents may be included in the formulation.

In another illustrative embodiment according to the invention, the particle separating agent treated hydrophobic active ingredient may be dispersed on an excipient such as microcrystalline cellulose, lactose, mannitol and any other excipient that is generally employed in oral dosage forms. The dispersed mixture can be filled in to a capsule or compressed into a tablet.

In another illustrative embodiment according to the invention, the particle separating agent treated hydrophobic active ingredient may be incorporated into a sustained release formulation. The excipient ensures better control over release profile and also complete release of the drug in the desired time interval.

In a further illustrative embodiment a solid pharmaceutical composition may be in the form of a multilayer system for oral administration. The system may be adapted to deliver two different actives such as the particle separating agent-treated hydrophobic active ingredient in one layer and hydrochlorothiazide in another layer.

In a further illustrative embodiment a solid pharmaceutical composition in the form of a multilayer system for oral administration is adapted to deliver an active pharmaceutical agent from a first layer immediately upon reaching the gastrointestinal tract, and to deliver a further pharmaceutical agent which may be same or different from a second layer, in a controlled manner over a specific time period.

The details of the invention, its objects and advantages are explained hereunder in greater detail in relation to non-limiting exemplary illustrations.

### EXAMPLES

### Example 1

### Formulation of Valsartan with Lutrol as Particle Separating Agent

A) Treatment of Valsartan with Poloxamer

TABLE 1
Composition of the particle separating agent-treated valsartan

| Ingredients | By weight ratio |
|---|---|
| Valsartan | 80.0 |
| Poloxamer 407 USP (Lutrol F127) | 16.0 |
| Microcrystalline Cellulose USP | 80.0 |
| (Avicel PH 102) | |

Valsartan was added to molten Lutrol F127 (at about 60-65° C.) under continuous mixing till a uniform semi-solid mixture was obtained. Weighed quantity of microcrystalline cellulose was added to the molten semi-solid mixture under continuous mixing. The blend was then allowed to cool to room temperature and the cooled mass was crushed and passed through 20# sieve.

TABLE 2
Comparative physical properties of valsartan and valsartan granules

| | | Vasartan treated with |
|---|---|---|
| Blend Properties | Valsartan | Lutrol |
| Bulk density (g/mL) | 0.12 | 0.393 |
| Angle of Repose | Could not be determined | 38.76 |
| Flow | as valsartan did not flow | Smoothly through 5 mm |
| | through the funnel | orifice |
| | despite tapping | |

By the treatment of valsartan with Lutrol, a free flowing granules were obtained which had excellent flow, increased bulk density and compressibility compared to valsartan itself.

B) Formulation of Lutrol-Treated Valsartan into Tablet (no Disintegrating Agent Added)

TABLE 3
Composition of tablet formulation

| Ingredients | Mg/tablet |
|---|---|
| Valsartan granules as prepared in part A | 176.0 |
| Microcrystalline Cellulose USP (Avicel PH 102) | 45.0 |
| Colloidal Silicon Dioxide USP (Aerosil 200) | 6.0 |
| Magnesium stearate USP | 3.0 |

A weighed quantity of valsartan granules were mixed with microcrystalline cellulose. The blend was further mixed with lubricants and compressed into tablets.

### Disintegration Test

Formulation was found to disintegrate within 3 minutes in 900 ml DM water. Disintegration test performed using USP disintegration apparatus at 37° C.

The tablet dosage form containing particle separating agent treated valsartan disintegrated rapidly despite absence of a traditional disintegrating agent. Further lutrol treated valsartan provided better processability compared to valsartan alone.

### Example 2

### Formulation of Untreated Valsartan and its Comparison with Lutrol Treated Valsartan

TABLE 4
Composition of untreated valsartan tablet formulation

| Ingredients | Mg/tablet |
|---|---|
| Valsartan | 80.0 |
| Microcrystalline Cellulose USP (Avicel PH 102) | 192.0 |
| Colloidal Silicon Dioxide USP (Aerosil 200) | 6.0 |
| Magnesium stearate USP | 3.0 |

Valsartan and microcrystalline cellulose was blended together, lubricated and compressed into tablets.

### Disintegration Test

Disintegration time is found to be highly hardness dependent. Therefore these comparisons were made at two different hardness levels.

TABLE 5
Comparison of disintegration time at different hardness levels

Disintegration
time (min)

| Hardness (N) | Example 2 | Example 1 |
|---|---|---|
| 43-58 | 4 | 0.66 |
| 78-100 | 12 | 2 |

Formulation comprising untreated valsartan was sticky in nature with poor processability. This resulted in problems of weight variation and sticking during compression.

It is evident from the above example that treatment with a particle separating agent not only improves processability of valsartan but also reduces the disintegration time of valsartan tablets.

### Example 3

### Formulation of Lutrol Treated Valsartan into Tablet with a Disintegrating Agent

A) Treatment of Valsartan with Lutrol

TABLE 6
Composition of the particle separating agent treated valsartan

| Ingredients | By weight ratio |
|---|---|
| Valsartan | 80.0 |
| Poloxamer 407 USP (Lutrol F127) | 16.0 |
| Microcrystalline Cellulose USP | 80.0 |
| (Avicel PH 102) | |

Valsartan was dry mixed with microcrystalline cellulose and the blend was granulated using molten Lutrol F127 (at about 60-65° C.) under continuous mixing The blend was then allowed to cool to room temperature and the cooled mass was crushed and passed through 20# sieve.

B) Formulation of Lutrol-Treated Valsartan into Tablet (Disintegrating Agent Added)

TABLE 7
Composition of tablet formulation

| Ingredients | Mg/tablet |
|---|---|
| Valsartan granules as prepared in part A | 176.0 |
| Crospovidone USP (Kollidon.CL) | 15.0 |
| Colloidal Silicon Dioxide USP (Aerosil 200) | 6.0 |
| Magnesium stearate USP | 3.0 |

A weighed quantity of valsartan granules were mixed with microcrystalline cellulose and crospovidone. The blend was further mixed with lubricants and compressed into tablets.

### Disintegration Test

Formulation was found to disintegrate within 1-2 minutes in, 900 ml DM water. Disintegration test performed using USP disintegration apparatus at 37° C.

Lutrol treated valsartan provided better processability compared to valsartan alone with a much reduced disintegration time.

C) Comparative In Vitro Dissolution Study in pH 6.8 phosphate buffer.

In-vitro dissolution studies were performed in pH 6.8 phosphate buffer media with following specifications:

### Dissolution Test Apparatus: USP Type II

### Temperature: 37.5±0.5° C.

### Rpm: 50

Sampling intervals: 15, 30 and 60 minutes
Sampling volume: 10 ml

TABLE 8
Comparative in vitro dissolution data in pH 6.8 buffer

| | Diovan | Example 3 |
|---|---|---|
| Time (minutes) | (% released) | (% released) |
| 0 | 0.0 | 0.0 |
| 15 | 92.2 | 73.6 |
| 30 | 97.6 | 97.6 |
| 60 | 98.0 | 102.8 |

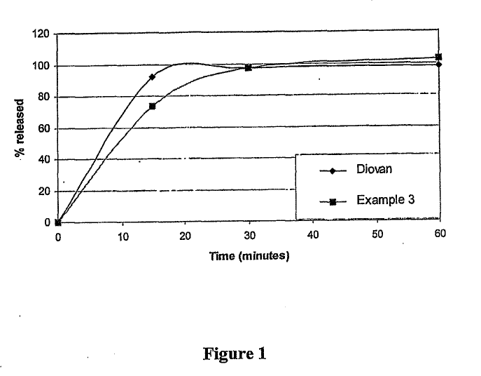

In-vitro dissolution data is shown in a graph in FIG. 1 of the accompanying drawing. The dissolution profile of the present invention in pH 6.8 buffer was found comparable to that of the innovator product.

### Example 4

### Effect of Treatment of Valsartan with a Particle Separating Agent on its Dissolution from Tablets in 0.1N HCl and its Comparison with Marketed Product Diovan® of Novartis

In-vitro dissolution studies were performed in 0.1N HCl with following specifications:

### Dissolution Test Apparatus: USP Type II

### Rpm: 75

### Temperature: 37.5±0.5° C.

Sampling intervals: 15, 30, 60 and 120 minutes
Sampling volume: 10 ml

TABLE 9
Comparative in vitro dissolution data in 0.1N HCl

| Time (minutes)/ | | |
|---|---|---|
| % Cum. Dissolved | Example 3 | Diovan ® |
| 0 | 0 | 0 |
| 15 | 6.3 | 4.1 |
| 30 | 12.9 | 10.7 |
| 60 | 20.4 | 18.3 |
| 120 | 31.3 | 29.4 |

Tablets of example 3 provide a dissolution rate comparable with that of the innovator product.

The innovator product contains a large amount of disintegrant but does not contain any particle separating agent. The treatment of valsartan with Lutrol resulted in similar disintegration time as that of innovator but more importantly the dissolution was found comparable under non-sink condition. This indicate that the treatment with a particle separating agent does not result in enhancement of solubility or dissolution rate as commonly observed with a wetting agent.

### Example 5

### In Vivo Bioequivalence Study

The study involved evaluation of in vivo performance of valsartan from the tablets in the invention as per example 3 (T) with respect to innovator product (R) i.e. Diovan® 80 in 8 healthy male volunteers under fasting condition in a complete crossover study. Pharmacokinetic parameters Cₘₐₓ (maximum plasma concentration), AUC₀₋ₜ (area under plasma concentration vs. time curve from 0 hours to the time of last sample collected) and AUC_{0-∞} (area under the plasma concentration vs. time curve from 0 hours to infinity) were calculated from the data.

TABLE 10
Summary statistics of pharmacokinetic parameters

| | Untransformed data | | |
|---|---|---|---|
| | Mean (C.V. %) | | |
| Parameters | Reference (R) | Test (T) | T/R ratio |
| Cₘₐₓ (ng/mL) | 3588.2 (32.7) | 3974.24 (38.99) | 1.107 |
| AUC₀₋ₜ (hr · ng/mL) | 22991.51 (38.64) | 26024.38 (39.91) | 1.131 |
| AUC_{0-∞} (hr · ng/mL) | 23601.49 (40.01) | 26457.70 (40.45) | 1.121 |
| Tₘₐₓ (hr) | 2.00 (34.55) | 2.66 (34.46) | |

Data indicate that formulation of example 3 is bioequivalent to Diovan®

### Example 6

### Formulation of Tablets of Valsartan Treated with Particle Separating Agent using Physical Mixing

A) Treatment of Valsartan with Lutrol

TABLE 11
Composition of the particle separating agent treated valsartan

| Ingredient | mg/tablet |
|---|---|
| Valsartan | 80 |
| Poloxamer 407 USP (Lutrol F127MP) | 8 |

A weighed quantity of valsartan was passed through 40# sieve and mixed with weighed quantity of Lutrol in planetary mixer at slow speed for a period of 30 minutes scraping the material every 10 minutes. Physical mixture obtained was passed through 30# sieve.

Physical mixing of valsartan with particle separating agent provided a reduced stickiness and static properties to valsartan.

B) Formulation of Lutrol-Treated Valsartan into Tablet

TABLE 12
Composition tablet formulation

| Ingredients | Mg/tablet |
|---|---|
| Lutrol treated valsartan as prepared in part A | 88.0 |
| Microcrystalline cellulose USP (Avicel PH 102) | 192.0 |
| Crospovidone USP (Kollidon.CL) | 40.0 |
| Colloidal Silicon Dioxide USP (Aerosil 200) | 9.0 |
| Magnesium stearate USP | 3.0 |

A weighed quantity of valsartan treated with Lutrol was mixed with microcrystalline cellulose and crospovidone. The blend was mixed with lubricants and compressed.

### Disintegration Test

Disintegration time was found to be 60 to 70 seconds.

### C) In-Vitro Dissolution Studies in pH 6.8 Phosphate Buffer

In-vitro dissolution studies were carried out with following specifications:

### Dissolution Test Apparatus: USP Type II

### Rpm: 50 Temperature: 37.5±0.5° C.

Sampling intervals: 10 and 20 minutes
Sampling volume: 10 ml

TABLE 13
In-vitro Dissolution studies

| Time (minutes) | % Cum. Dissolved |
|---|---|
| 0 | 0 |
| 10 | 91.9 |
| 20 | 101.3 |

It is evident from the above examples that the formulation as per the present invention involves a simple manufacturing process and provides good processability, faster disintegration and desired dissolution rate.

### Example 7

### Formulation of Tablets of Atorvastatin Treated with Stearoyl Macrogol Glycerides (Gelucire 50/13)

A) Treatment of Atorvastatin with Gelucire (50/13)

TABLE 14
Composition of particle separating agent treated Atorvastatin

| Ingredients | Mg/tablet |
|---|---|
| Atorvastatin calcium | 86.75 |
| Stearoyl Macrogol Glycerides USP (Gelucire 50/13) | 68.8 |
| Microcrystalline Cellulose USP (Avicel pH 102) | 215.0 |
| Lactose Monohydrate (Pharmatose 200M) | 100.0 |

Atorvastatin calcium, microcrystalline cellulose and lactose were dry mixed. The blend was granulated using molten Gelucire 50/13 (at about 50° C.) and cooled to room temperature. The cooled mass was passed through 20# sieve.

By treatment of atorvastatin with Gelucire (50/13), free flowing granules were obtained which had an excellent flow and compressibility.

B) Formulation of Gelucire-Treated Atorvastatin into Tablet

TABLE 15
Composition of tablet formulation

| Ingredients | Mg/tablet |
|---|---|
| Atorvastatin granules as prepared in part A | 470.55 |
| Microcrystalline Cellulose USP (Avicel pH 102) | 159.45 |
| Lactose Monohydrate (Pharmatose 200M) | 100.0 |
| Colloidal silicon dioxide USP (Aerosil 200) | 10 |
| Magnesium stearate USP | 5.0 |

Weighed quantity of granules were mixed with microcrystalline cellulose (# 40) and lactose for a period of 5 minutes, lubricated and compressed.

### Disintegration Test:

The formulation was found to disintegrate within less than 60 sec in 900 ml distilled water.

### Example 8

### Formulation of Atorvastatin with Polyethylene Glycol 660 Hydroxystearate (Solutol HS15) as Particle Separating Agent

A) Treatment of Atorvastatin with Solutol HS15

TABLE 16
Composition of the particle separating agent treated atorvastatin

| Ingredients | Mg/tablet |
|---|---|
| Atorvastatin calcium | 86.75 |
| Polyethylene glycol 660 hydroxystearate (Solutol HS15) | 120.0 |
| Calcium carbonate | 54.0 |
| L-arginine | 186.0 |
| Microcrystalline Cellulose USP (Avicel pH 102) | 270.0 |
| Lactose Monohydrate (Pharmatose 200M) | 90.24 |

Weighed quantities of atorvastatin calcium, calcium carbonate, arginine, microcrystalline cellulose and lactose were dry mixed. Molten Solutol HS15 was added to the dry mix under continuous mixing. The mass was stirred and sheared till it attained room temperature. The cooled mass was passed through 20# sieve.

By treatment of atorvastatin with Solutol HS15, free flowing granules were obtained which had excellent flow and compressibility.

B) Formulation of Solutol-Treated Atorvastatin into Tablet

TABLE 17
Composition of tablet formulation

| Ingredients | Mg/tablet |
|---|---|
| Atorvastatin granules as prepared in part A | 807.0 |
| Microcrystalline Cellulose USP (Avicel pH 102) | 284.0 |
| Lactose Monohydrate (Pharmatose 200M) | 97.0 |
| Magnesium stearate USP | 12.0 |

Weighed quantity of granules was mixed with microcrystalline cellulose and lactose for a period of 5 minutes. The blend was further mixed with lubricants and compressed.

Disintegration Test:

The formulation was found to disintegrate within 20 seconds in 900 mL distilled water.

### Example 9

### Treatment of Candesartan with Solutol HS15 Particle Separating Agent

A) Treatment of Candesartan with Solutol HS15

TABLE 18
Composition of particle separating agent treated candesartan

| Ingredients | Mg/tablet |
|---|---|
| Candesartan | 16.0 |
| Polyethylene glycol 660 hydroxystearate (Solutol HS15) | 12.0 |
| Microcrystalline Cellulose USP (Avicel pH 102) | 32.0 |

Weighed quantities of Candesartan and Avicel PH102 were dry mixed. Molten Solutol HS15 was added to the dry mix under continuous mixing. The mass was stirred and sheared till it attained room temperature. The cooled mass was passed through 20# sieve.

By treatment of candesartan with solutol, a free flowing mass was obtained which had excellent flow and compressibility.

B) Formulation of Solutol HS15-Treated Candesartan into Tablet

TABLE 19
Composition of tablet formulation

| Ingredients | Mg/tablet |
|---|---|
| Candesartan granules as prepared in Part A | 60.0 |
| Microcrystalline Cellulose USP (Avicel pH 102) | 50.0 |
| Colloidal silicon dioxide USP (Aerosil 200) | 6.0 |
| Magnesium stearate USP | 3.0 |

Weighed quantity of granules were mixed with microcrystalline cellulose for a period of 5 minutes. The blend was further mixed with lubricants and compressed.

### Disintegration Test:

The formulation was found to disintegrate within less than 3 minutes in 900 ml distilled water.

### Example 10

### Formulation of Irbesartan with Solutol HS15 as Particle Separating Agent

A) Treatment of Irbesartan with Particle Separating Agent

TABLE 20
Composition of particle separating agent treated irbesartan

| Ingredients | Mg/tablet |
|---|---|
| Irbesartan | 75.0 |
| Polyethylene glycol 660 hydroxystearate (Solutol HS15) | 15.0 |
| Microcrystalline Cellulose USP (Avicel pH 102) | 40.0 |

Weighed quantities of irbesartan and microcrystalline cellulose were dry mixed. Molten Solutol HS15 was added to dry mix under continuous mixing sheared till it attained room temperature. The cooled mass was passed through 20# sieve.

By treatment of irbesartan with Solutol HS15 free flowing granules were obtained which has excellent flow and compressibility.

B) Formulation of Solutol HS15-Treated Irbesartan into Tablet

TABLE 21
Composition of tablet formulation containing Irbesartan

| Ingredients | Mg/tablet |
|---|---|
| Irbesartan granules as prepared in part A | 130.0 |
| Microcrystalline Cellulose USP (Avicel pH 102) | 25.0 |
| Colloidal silicon dioxide USP (Aerosil 200) | 6.0 |
| Magnesium stearate USP | 3.0 |

Weighed quantity of granules were mixed with microcrystalline cellulose for a period of 5 minutes. The blend was mixed with lubricants for a period of 2 minutes and compressed.

### Disintegration Test:

The formulation was found to disintegrate within less than 120 sec in 900 ml distilled water.

### C) In Vitro Dissolution in 0.1N HCl:

In-vitro dissolution studies were carried out with following specifications:

### Dissolution Test Apparatus: USP Type II,

### Rpm: 50

### Temperature: 37.5±0.5° C.

Sampling intervals: 30 minutes
More than 80% of irbesartan was released at the end of 30 minutes in 0.1 N HCl.

1. A novel solid oral dosage form comprising a therapeutically effective amount of hydrophobic pharmacological active ingredient and at least one particle separating agent.

2. The novel solid oral dosage form according to claim 1, comprising a therapeutically effective amount of hydrophobic pharmacological active ingredient and at least one particle separating agent, prepared without a disintegrating agent.

3. The novel solid oral dosage form according to claim 1, comprising a therapeutically effective amount of hydrophobic pharmacological active ingredient and at least one particle separating agent, prepared with a minimum amount of a disintegrating agent.

4. The novel solid oral dosage form according to claim 1 wherein the particle separating agent is selected from the class of wetting agent(s)

5. The novel solid oral dosage form according to claim 4 wherein the said wetting agent(s) is selected from hydrophilic surfactants or lipophilic surfactants or mixtures thereof.

6. The novel solid oral dosage form according to claim 5 wherein the said surfactants are selected from anionic, nonionic, cationic, and amphiphilic surfactants.

7. The novel solid oral dosage form according to claim 5 wherein the said wetting agent(s) is selected from PEG-20-glyceryl stearate, PEG-40 hydrogenated castor oil, PEG 6 corn oil, lauryl macrogol-32 glyceride stearoyl macrogol glyceride, polyglyceryl-10 mono dioleate, Propylene glycol dioctanoate, Propylene glycol caprylate/caprate, Glyceryl monooleate, Glycerol monolinoleat, Glycerol monostearate, PEG-20 sorbitan monolaurate, PEG-4 lauryl ether, Sucrose distearate, Sucrose monopalmitate, polyoxyethylene-polyoxypropylene block copolymer, polyethylene glycol 660 hydroxystearate, Sodium lauryl sulphate, Sodium dodecyl sulphate, Dioctyl suphosuccinate, L-hydroxypropyl cellulose, hydroxylethylcellulose, hydroxy propylcellulose, Propylene glycol alginate, sodium taurocholate, sodium glycocholate, sodium deoxycholate, betains, polyethylene glycol, d-α-tocopheryl polyethylene glycol 1000 succinate and mixtures thereof.

8. The novel solid oral dosage form according to claim 7 wherein the said wetting agent(s) is stearoyl macrogol glyceride, polyoxyethylene-polyoxypropylene block copolymer, polyethylene glycol 660 hydroxystearate, Sodium lauryl sulphate, polyethylene glycol, d-α-tocopheryl polyethylene glycol 1000 succinate and mixtures thereof.

9. The novel solid oral dosage form according to claim 1 wherein the said hydrophobic pharmacological active ingredient belongs to the class of angiotensin receptor blocking agents.

10. The novel solid oral dosage form according to claim 9 wherein the said hydrophobic pharmacological active ingredient is valsartan.

11. The novel solid oral dosage form according to claim 1 wherein the said hydrophobic pharmacological active ingredient is a combination of valsartan and hydrochlorothiazide.

12. The novel solid oral dosage form according to claim 1 wherein the said hydrophobic pharmacological active ingredient belongs to the class of 3-hydroxy-3-methylglutaryl coenzyme A (HMG CoA) reductase inhibitors.

13. The novel solid oral dosage form according to claim 1 wherein the said hydrophobic pharmacological active ingredient is atorvastatin.

14. The novel solid oral dosage form according to claim 1 wherein the ratio of hydrophobic active ingredient to particle separating agent is about 20:1 to about 1:20.

15. The novel solid oral dosage form according to claim 1 wherein the ratio of hydrophobic active ingredient to particle separating agent is preferably about 10:1 to about 1:10.

16. The novel solid oral dosage form according to claim 1 wherein the ratio of hydrophobic active ingredient to particle separating agent is most preferably about 5:1 to about 1:5.

17. A process for the preparation of the novel solid oral dosage form comprising,
(a) treating a hydrophobic active ingredient with at least one particle separating agent, and
(b) incorporating the treated hydrophobic active ingredient into a solid dosage form.

18. The process according to claim 17 comprising treating a hydrophobic active ingredient with at least one particle separating agent using melt granulation, solvent treatment or physical mixing processes.

19. The process according to claim 18 comprising treating a hydrophobic active ingredient with at least one particle separating agent using melt granulation process.

20. The process according to claim 17 wherein the said hydrophobic pharmacological active ingredient belongs to the class of angiotensin receptor blocking agents.

21. The process according to claim 20 wherein the said hydrophobic pharmacological active ingredient is valsartan.

22. The process according to claim 17 wherein the said hydrophobic pharmacological active ingredient belongs to the class of 3-hydroxy-3-methylglutaryl coenzyme A (HMG CoA) reductase inhibitors.

23. The novel solid oral dosage form according to claim 1 wherein the said dosage form is tablet, capsule, pellet, granule or powder.

24. The process according to claim 17 wherein the said dosage form is tablet.

25. The novel solid oral dosage form according to claim 1 further comprises binder, disintegrant, basifying agent, lubricant and diluent.

26. The novel solid oral dosage form according to claim 24, wherein the dosage form is prepared by wet granulation, direct compression, dry granulation or molding method.

27. The novel solid oral dosage form according to claim 1, wherein the said dosage form is coated.

28. The novel solid oral dosage form according to claim 27 wherein the said coated tablet comprises coat in the form of quick dissolving film of polymer selected from the group of Hydroxypropylmethyl cellulose, Hydroxypropyl cellulose, Carboxymethyl Cellulose, polyvinyl alcohol, poly methacrylate and the like.

29. The novel solid oral dosage form according to claim 27, wherein the said coat is functional coat.

30. The novel solid oral dosage form according to claim 29, wherein the said functional coat comprises polymer selected from the group comprising of hydrophilic polymers, hydrophobic polymers, waxes and the like.

31. The novel solid oral dosage form according to claim 1, wherein the said dosage form is multilayered tablet.

32. A novel oral solid dosage form comprising valsartan and at least one particle separating agent.

33. A novel oral solid dosage form comprising atorvastatin and at least one particle separating agent.
